SEQUENCE LISTING (5 sequences)

SEQ ID NO: 1 - protein, 24 aa, Artificial Sequence
  ACTH
SYSMEHFRWGKPVGKKRRPVKVYP

SEQ ID NO: 2 - protein, 154 aa, Artificial Sequence
  myoglobin
MGLSDGEWQQVLNVWGKVEADIAGHGQEVLIRLFTGHPETLEKFDKFKHLKTEAEMKASEDLKKHGTVVLTALGGILKKKGHHEAELKPLAQSHATKHKIPIKYLEFISDAIIHVLHSKHPGDFGADAQGAMTKALELFRNDIAAKYKELGFQG

SEQ ID NO: 3 - protein, 105 aa, Artificial Sequence
  cytochrome c
MGDVEKGKKIFVQKCAQCHTVEKGGKHKTGPNLHGLFGRKTGQAPGFSYTDANKNKGITWGEETLMEYLENPKKYIPGTKMIFAGIKKKGEREDLIAYLKKATNE

SEQ ID NO: 4 - protein, 214 aa, Artificial Sequence
  AS1-Casein
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 5 - protein, 12 aa, Artificial Sequence
  synthetic peptide
LYAADTRLYAVR

(19) United States
(12) Patent Application Publication
(10) Pub. No.: US 2022/0120758 A1
(43) Pub. Date: Apr. 21, 2022

(54) METHOD AND DEVICE FOR PROTEIN SEQUENCE ANALYSIS

(71) Applicant: FUDAN UNIVERSITY, Shanghai (CN)

(72) Inventors: Richard ZARE, Shanghai (CN); Xiaoqin ZHONG, Shanghai (CN)

(73) Assignee: FUDAN UNIVERSITY, Shanghai (CN)

(21) Appl. No.: 17/424,124

(22) PCT Filed: Jun. 6, 2019
(86) PCT No.: PCT/CN2019/090391

§ 371 Date: Jul. 19, 2021

(51) Int. Cl. G01N 33/68

(52) U.S. Cl. CPC G01N 33/6848; G01N 33/6821

(57) ABSTRACT

A method and a device for protein sequence analysis, and the use of microdroplets for improving protein sequencing by accelerating enzymatic digestion, wherein the method comprises the following steps: a) forming a solution containing protein into microdroplets having a size small enough to result in acceleration of protein digestion; b) introducing the microdroplets into a mass spectrometer (MS) for real-time detection; c) obtaining analysis result of the protein from the mass spectrometer (MS); wherein the protein is fully digested in the microdroplets before entering the mass spectrometer (MS). The method and device can achieve simple and nearly complete protein digestion in a very short time and obtain high sequence coverage.

### TECHNICAL FIELD

The present invention relates to a method and a device for protein sequence analysis. More particularly, the present invention relates to a method of protein sequence analysis and a microdroplet-MS (microdroplet-mass spectrometer) device for protein sequence analysis. The present invention also relates to the use of microdroplets for improving protein sequencing by accelerating enzymatic digestion, wherein the microdroplets are formed from protein-containing solution and have a size small enough to result in acceleration of protein digestion.

### BACKGROUND ART

In bottom-up proteomics, enzymatic digestion of proteins is an essential and critical approach for breaking down proteins into smaller polypeptides prior to analysis for protein structure elucidation by mass spectrometry (MS).¹ In a typical enzymatic digestion process, the protein solution is mixed with a proper amount of enzyme, such as trypsin, and incubated overnight at 37° C. However, such process is time-consuming. To facilitate digestion, protein denaturation is usually performed before the digestion to destroy the compact, globular structure and expose more proteolytic cleavage sites. Methods commonly used for protein denaturation include the application of external stress or additives, such as heat, radiation, or urea. In addition, reductive alkylation is often used to remove disulfide bonds. To further accelerate protein digestion, various attempts have been taken to reduce the digestion time from overnight to several minutes, including increasing the digestion temperature, using columns or porous materials for trypsin immobilization, addition of organic solvents, applying microwave energy or focused ultrasonic field, or a combination of any thereof.² Still, the conventional methods for sequence analysis of protein require pre-treatment steps prior to enzymatic digestion, which is relatively time consuming and inconvenient.

The most ideal case in enzymatic digestion for proteomics study is achieved when all cleavage sites are digested, but in practice, enzymes often fail to cleave all scissile bonds, even though the reaction time is sufficiently long. This failure to achieve complete coverage is mainly attributed to neighboring amino acids around the cleavage sites. The presence of acidic residues, glutamate (E) or aspartate (D), near the cleavage site was reported to reduce the proteolysis speed significantly by forming salt bridges with the basic arginine (R) and lysine (K) and inhibiting the approach of R or K to the complementary aspartic acid at the bottom of the trypsin active site.

In the past few years, microdroplets have been extensively reported to accelerate dramatically various kinds of single-phase or two-phase organic reactions with slow kinetics or assisted by specific catalysis in the bulk phase. Many explanations for this behavior have been advanced, some of the earliest involving reagent concentration, which is increased by droplets undergoing evaporation and fission. Microdroplet chemistry has also been used to capture and identify transient reaction intermediates when combined with online MS analysis, to study the fast reaction kinetics via microdroplet fusion MS, to perform preparative syntheses, and also to facilitate material synthesis. A number of setups were created to generate micron-size droplets (microdroplets) including microfluidics, surface drop-casting, or different spray-based ionization methods.³ The exact reasons for the reaction rate acceleration in microdroplets are still not clearly established, but it is commonly accepted that it is mainly caused by the striking difference between the environments of microdroplets and the corresponding bulk phase. Various factors may contribute, such as droplet size, surface charge, reagent confinement, solvent composition, and droplet evaporation. In addition to the achievement in organic synthesis, microdroplets are of interest in promoting biochemical reactions because of the gentleness of the process and particularly because aqueous microdroplets provide a benign environment that is compatible with life. However, the application of microdroplet to biochemical analysis has seldom been investigated.

### SUMMARY OF INVENTION

### Technical Problem

Conventional methods or devices for protein sequence analysis require a pretreatment step of enzymatic digestion, which is relatively time consuming and inconvenient. Moreover, enzymes often fail to cleave all scissile bonds, even though the reaction time is sufficiently long. Therefore, there is a need in the art to reduce the digestion time of proteins and improve sequence coverage for protein identification.

The present invention aims to solve the above problems and provide a method and a device for protein sequence analysis.

### Solution to Problem

Inventors of this invention surprisingly find that, microdroplets could remarkable accelerate the proteolysis speed despite of the negative influence of acidic microdroplets on enzymatic digestion, and that the digestion rate markedly increases as the size of the aqueous microdroplet shrinks. Based on this, microdroplets can be used as a simple, ultrafast, and powerful tool for protein analysis when coupled directly with a mass spectrometer (MS).

The present invention therefore relates to a method of protein sequence analysis, comprising the following steps: a) forming a solution containing protein into microdroplets having a size small enough to result in acceleration of protein digestion; b) introducing the microdroplets into a mass spectrometer (MS) for real-time detection; c) obtaining analysis result of the protein from the mass spectrometer (MS); wherein the protein is fully digested in the microdroplets before entering the mass spectrometer (MS).

Further, the method of the invention does not require any pre-treatment step for enzymatic digestion.

Further, according to the method of the invention, the protein is recalcitrant to tryptic digestion.

Further, according to the method of the invention, the pH value of the solution containing protein is in the range of 4˜11.

Further, according to the method of the invention, the pH value of the solution containing protein is about 8.

Further, according to the method of the invention, in step a), the microdroplets are formed by a spray-based ionization method.

Further, according to the method of the invention, in step a), the microdroplets are formed by means of capillary of a sprayer.

Further, according to the method of the invention, the sprayer is applied with pressurized nebulizing N₂ gas at a pressure of 120 psi or more.

Further, according to the method of the invention, the sprayer is applied with a positive or negative voltage of 3 kV or above.

Further, according to the method of the invention, the microdroplets travel a distance of 10 mm to 10 cm before entering the mass spectrometer.

Further, according to the method of the invention, the microdroplets travel a distance of 50 mm before entering the mass spectrometer.

Further, according to the method of the invention, the microdroplets travel a distance of 2 cm before entering the mass spectrometer.

In another aspect, the present invention relates to a microdroplet-MS device for protein sequence analysis, comprising: a microdroplet-producing unit, and a mass spectrometer (MS) unit that is directly coupled with the microdroplet-producing unit, wherein the microdroplet-producing unit forms a protein-containing solution into microdroplets having a size small enough to result in acceleration of protein digestion.

Further, according to the microdroplet-MS device of the invention, the microdroplet-producing unit includes a sprayer.

Further, according to the microdroplet-MS device of the invention, the sprayer is provided with a capillary having an inner diameter of 50˜100 μm.

Further, according to the microdroplet-MS device of the invention, the sprayer is applied with a positive or negative voltage of 3 kV or above.

Further, according to the microdroplet-MS device of the invention, the travel distance of microdroplets from the sprayer to the mass spectrometer is in the range of 10 mm to 10 cm.

Further, according to the microdroplet-MS device of the invention, the travel distance of microdroplets from the sprayer to the mass spectrometer is 50 mm.

Further, according to the microdroplet-MS device of the invention, the travel distance of microdroplets from the sprayer to the mass spectrometer is 2 cm.

In another aspect, the present invention relates to the use of microdroplets for improving protein sequencing by accelerating enzymatic digestion, wherein the microdroplets are formed from protein-containing solution and have a size small enough to result in acceleration of protein digestion.

### Advantageous Effects of Invention

The present invention relates to a method of protein sequence analysis, comprising the following steps: a) forming a solution containing protein into microdroplets having a size small enough to result in acceleration of protein digestion; b) introducing the microdroplets into a mass spectrometer (MS) for real-time detection; c) obtaining analysis result of the protein from the mass spectrometer (MS); wherein the protein is fully digested in the microdroplets before entering the mass spectrometer (MS). By using room-temperature microdroplet chemistry, the method of the invention achieves simple and nearly complete protein digestion in a very short time (e.g., less than 1 ms).

The present invention also relates to a microdroplet-MS device for protein sequence analysis, comprising: a microdroplet-producing unit, and a mass spectrometer (MS) unit that is directly coupled with the microdroplet-producing unit, wherein the microdroplet-producing unit forms a protein-containing solution into microdroplets having a size small enough to result in acceleration of protein digestion. The microdroplet-MS device according to the invention provides a convenient interface to directly couple the sample separation with MS for sequential digestion and online analysis of trace amount of protein mixture, and the microdroplet-producing unit therein also acts as a MS emitter.

The method and device of the invention achieve simple and nearly complete protein digestion in a very short time and obtain high sequence coverage. Surprisingly, the microdroplets generated during electrosonic spray ionization (ESSI) and directly coupled with a mass spectrometer (microdroplet-MS) could realize online digestion of relatively large peptides. Thus, the method and device for protein sequence analysis according to the invention does not require any pre-treatment step for enzymatic digestion.

It is demonstrated herein that microdroplet is a practical and nearly universal technique for protein sequencing. In particular, microdroplet-MS is able to markedly accelerate the digestion of proteins, even those that have proven to be particularly recalcitrant to tryptic digestion. Thus, the method and device of the invention is suitable for the sequence analysis of a wide variety of proteins.

### BRIEF DESCRIPTION OF DRAWINGS

FIG. 1. Schematic of the experimental apparatus for the online proteolysis by microdroplet chemistry coupled with mass spectrometry (microdroplet-MS). The inner capillary has an i.d. of 50 μm and an o.d. of 148 μm to which a high voltage source is connected.

FIG. 2. Mass spectra of 10-μM human ACTH (1-24) in 5-mM NH₄HCO₃ sprayed by the homemade sprayer: (a1) undigested (no trypsin); (a2-a5) digested with 5-μg/mL trypsin different travel distances between the sprayer tip and MS inlet for 2, 10, 20, and 50 mm, respectively. * denotes the peptide fragments, and # marks undigested ACTH peaks. (b) 26 peptide fragments from the digestion of human ACTH (1-24). The sequence is SYSMEHFR|WGKPVGK|K|R|RPVK|VYP, where vertical lines have been added where trypsin digestion is expected to occur. (c) Variation with microdroplet travel distance between the sprayer tip and the MS inlet of the ratio of the intensity of a chosen peptide at m/z 632.4 marked in pink in FIG. 2 to the sum of the intensities of all peaks from intact multicharged ACTH.

FIG. 3. MS/MS-CID spectrum of the peptide at m/z 632.4 from 10 μM ACTH digest in 5 mM NH₄HCO₃ by microdroplet-MS with a normalized energy of 25 and the isolated width of 1 m/z.

FIG. 4. Comparison of ACTH digestion with various methods: (a) standard ESI-MS, (b) bulk phase at 37° C. for 3 h, followed by analysis with standard ESI-MS. (c) The 16 peptide peaks found by bulk phase digestion of human ACTH (1-24) for 3 h at 37° C. * denotes the peptide fragments, and # marks undigested ACTH peaks.

FIG. 5. Mass spectra of myoglobin digestion with various methods: (a) standard ESI-MS, (b) bulk phase at 37° C. for 14 h, followed by analysis with standard ESI-MS, and microdroplet MS applied with (c) positive high voltage at +3 kV, and (d) negative high voltage at −3 kV.

FIG. 6. Peptides identified from digests of myoglobin by microdroplet-MS and standard MS applied with a positive voltage.

FIG. 7. Peptides identified from digests of myoglobin by microdroplet-MS applied with a negative voltage.

FIG. 8. Peptides identified from digests of cytochrome c by microdroplet-MS applied with a positive voltage.

FIG. 9. (a) Peptide sequence, (b) mass spectrum of pure peptide, and mass spectra of the peptide digested with various methods: (c) standard ESI-MS, (d) bulk phase at 37° C. for 1 h, followed by analysis with standard ESI-MS, and (e) microdroplet-MS applied with a high voltage of +3 kV.

FIG. 10. Mass spectra of the digest of a synthetic peptide in 5 mM NH₄HCO₃ with different pH values by microdroplet-MS applied with a positive voltage.

FIG. 11. Mass spectra showing the trypsin microdroplet digestion of (a) α-casein and (b) cytochrome c at a positive voltage of +3 kV. (c) PAGE gel showing the two protein bands stained with Coomassie blue. Red asterisks denote the peptide fragments.

FIG. 12. Peptides identified from digests of cytochrome c and α-casein extracted from a SDS-PAGE gel by microdroplet MS applied with a positive voltage.

### DESCRIPTION OF THE INVENTION

Before any embodiments of the disclosure are explained in detail, it is to be understood that the invention is not limited in its application to the details of construction and the arrangement of components set forth in the following description or illustrated in the figures and examples. The section headings used herein are for organizational purposes only and are not to be construed as limiting the subject matter described. All references cited in this application are expressly incorporated by reference herein for all purposes.

The disclosure embraces other embodiments and is practiced or carried out in various ways. Also, it is to be understood that the phraseology and terminology used herein is for the purpose of description and should not be regarded as limiting.

The term “electrosonic spray ionization (ESSI)” used herein refers to an ionization technique that combines electrospray ionization (ESI) and sonic spray ionization (SSI).

The term “collision-induced dissociation (CID)” used herein refers to a tandem mass spectrometry technique to induce fragmentation of selected ions by colliding with gas phase.

The term “adrenocorticotropic hormone (ACTH)” used herein refers to a polypeptide consisting of 39 amino acids and produced by the front of the pituitary gland in the brain. The function of ACTH is to regulate levels of the steroid hormone cortisol released from the adrenal gland.

The term “myoglobin” used herein refers to a protein containing 153 amino acid residues and a heme group with iron at its center. Myoglobin (symbol Mb or MB) is an iron- and oxygen-binding protein found in the muscle tissue of vertebrates in general and in almost all mammals. Myoglobin has proven to be recalcitrant to tryptic digestion.

The method of protein sequence analysis of the present invention comprises the following steps: a) forming a solution containing protein into microdroplets having a size small enough to result in acceleration of protein digestion; b) introducing the microdroplets into a mass spectrometer (MS) for real-time detection; c) obtaining analysis result of the protein from the mass spectrometer (MS); wherein the protein is fully digested in the microdroplets before entering the mass spectrometer (MS).

The microdroplet-MS device for protein sequence analysis of the present invention comprises: a microdroplet-producing unit, and a mass spectrometer (MS) unit that is directly coupled with the microdroplet-producing unit, wherein the microdroplet-producing unit forms a protein-containing solution into microdroplets having a size small enough to result in acceleration of protein digestion. The microdroplet-MS device of the invention is a useful tool for sequence analysis of proteins. In particular, microdroplet-MS is a powerful proteolysis tool that could easily cleave most theoretical-scissile bonds and produce less missed cleavage peptides.

In bottom-up proteomics, sample preparation, including protein digestion, is a lengthy step and remains the bottleneck in terms of time. Table 1 lists various approaches for accelerating the protein digestion.

TABLE 1
Comparisons of various techniques
for accelerating protein digestion.

| Accelerated technique | Online | Digestion time | |
|---|---|---|---|
| High temperature | possible | ~15 | min |
| Microwave | possible | ≤15 | min |
| Ultrasound | Not feasible | ≤5 | min |
| High pressure | Yes | <1 | min |
| Infrared | Not done | ~5 | min |
| Organic solvent | Not done | ≤5 | h |
| On-column immobilized enzyme | Yes | <6 | min |
| On-chip immobilized enzyme | Yes | 5 | s |
| Magnetic particle immobilized enzyme | Yes | ~30 | s |
| Microdroplets (this work) | Yes | <1 | ms |

Inventors of this invention surprisingly find that the digestion rate markedly increases as the size of the aqueous microdroplet shrinks. This finding suggests that protein bond cleavage occurs at or near the air-water interface because the ratio of the droplet surface to droplet volume increases as the diameter of the droplet decreases.

As shown in Table 1, according to the invention, microdroplets coupled with mass spectrometer (MS) gives the shortest digestion time (less than 1 ms). In addition, digestion in microdroplets consumes only a very small amount of samples (0.16 μL/s) and could be easily extended to other setups that produce microdroplets.

Microdroplets can be produced by various spray-based ionization methods.

In preferred embodiments, in order to achieve better acceleration of enzymatic digestion while ensuring a good MS signal, a capillary having an inner diameter within a certain range is applied to obtain desired microdroplets.

In preferred embodiments, the microdroplets are produced by a sprayer with a capillary having an inner diameter of 50˜100 μm. If the size is too big, the generated droplet is too big and the acceleration effect will be reduced. If the size is too small, the sample infusion may be not continuously, which will make the MS signal worse. In an embodiment, the capillary has an inner diameter of 60 μm, 70 μm, 80 μm, or 90 μm.

Further, in order to achieve better acceleration of enzymatic digestion, the pressure of nebulizing N₂ gas applied to the sprayer is increased to obtain smaller microdroplets.

In a particularly preferred embodiment, the sprayer is applied with pressurized nebulizing N₂ gas at a pressure of 120 psi or more, preferably 130 psi or more, more preferably 140 psi or more, yet more preferably 150 psi or more, still more preferably 200 psi or more.

It should be appreciated that the temperature of the mass spectrometer inlet and the polarity of the voltage applied to the spray capillary had little effect on the digestion process, although a negative potential is superior to a positive potential. Even no potential being applied is also effective, but less so.

In preferred embodiments, microdroplets containing the protein and enzyme are generated by electrosonic spray in which a sheath of rapidly flowing dry N₂ gas surrounds a capillary held at typically −3 kV.

In preferred embodiments, to sequence the peptide of interest, collision-induced dissociation (CID) is applied for the fragmentation of the isolated precursor ion with an isolation width of 1 m/z and optimized collision energy of 25 under full scan mode.

In preferred embodiments, the protein sequence was acquired from the UniprotKB database with its specific accession numbers. The peptide sequences are identified by comparison of observed peptide molecular weights with theoretical ones using MS-digest program from Protein Prospector version 5.19.1 (University of California, San Francisco, Calif., USA) to perform an in silico digest of the protein of interest.

Once inside the heated inlet, the microdroplet evaporates and the reaction stops. Therefore, the digestion time in the microdroplet can be determined by the travel distance between the sprayer tip and the MS inlet. Moreover, the digestion efficiency is directly correlated with the reaction time in microdroplet. Therefore, the digestion yield can be improved by increasing the travel distance.

In preferred embodiments, the travel distance of microdroplets from the sprayer to the mass spectrometer is at least 10 mm, preferably in the range of 10 mm to 10 cm, more preferably 20 mm, yet more preferably 50 mm, still more preferably 2 cm. When the travel distance is 10 mm or more, fragments covering the whole sequence of a polypeptide can be successfully identified according to the method of the invention. When the travel distance is 50 mm or more, almost complete digestion of relatively large polypeptides can be achieved according to the method of the invention.

In preferred embodiments, the pH value of the solution containing protein for microdroplet-MS analysis is more than 4, preferably in the range of 4˜11, more preferably is about 8. A pH value in the range of 4˜11 allows microdroplet-MS to maintain excellent acceleration effect for the proteolysis.

In preferred embodiments, the method and device of the invention is used for digestion and analysis of proteins, in particular those that have proven to be particularly recalcitrant to tryptic digestion. By using the method and device of the invention, the time for digestion of proteins that have proven to be particularly recalcitrant to tryptic digestion could be dramatically reduced, for example, from overnight to less than 1 ms.

In certain embodiments, proteins include, but are not limited to, adrenocorticotropic hormone (ACTH), myoglobin, cytochrome c, BSA, hemoglobin, beta-lactoglobulin, casein and other common proteins known in the art, as well as synthetic peptides.

Aqueous microdroplet can be used as a simple, ultrafast, and powerful tool for protein analysis when coupled directly with MS. The proteolysis-resistant proteins, such as myoglobin, may be fully digested to obtain high sequence coverage and complete cleavage of theoretically cleavable peptide bonds, indicating the great advance of the unique environment provided by microdroplet for sample mixing and protein structure alteration.

Accordingly, the present invention also relates to the use of microdroplets for improving protein sequencing by accelerating enzymatic digestion, wherein the microdroplets are formed from protein-containing solution and have a size small enough to result in acceleration of protein digestion.

### EXAMPLES

The present invention will be specifically described with reference to, but not limited to, examples.

Ammonia bicarbonate and all the protein reagents used are obtained from Sigma-Aldrich (Shanghai, China). Deionized water (18.2 MΩ cm) is prepared by the Milli Q purification system (Millipore Advantage A10) and used in all aqueous solutions.

### Example 1

Optimization of the Performance of Microdroplet-MS.

To optimize of the performance of microdroplet-MS, tryptic digestion of ACTH was used as a simple model system.

A stream of microdroplets was generated by infusing an aqueous sample solution containing 10 μM adrenocorticotropic hormone from human (ACTH, 1-24, Genscript, China) and 5 μg/mL trypsin in 5 mM ammonia bicarbonate (NH₄HCO₃, pH 8) with a syringe at a flow rate of 10 μL/min into a homemade sprayer (with a capillary of 50 μm i.d and 148 μm o.d, as shown in FIG. 1).

The sample solution was sprayed from the tip of the fused silica capillary (148 μm o.d., 50 μm i.d., Polymicro Technologies, China) and assisted by a nebulizing gas of dry N₂ with a pressure of 120 psi. By placing the sprayer in front of a high-resolution mass spectrometer (LTQ Orbitrap Elite, Thermo Scientific, San Jose, Calif.) at a proper position, the microdroplets were directed into MS for real-time detection. The MS inlet capillary was maintained at 275° C. and capillary voltage at 0 V. No other source gases were used when digestion was performed in microdroplets.

The droplet size was estimated to be around 6 μm in diameter. The microdroplets travelled in the air at a speed of 84±18 m/s. The digestion time in the microdroplets were determined by the travel distance between the sprayer tip and the MS inlet.

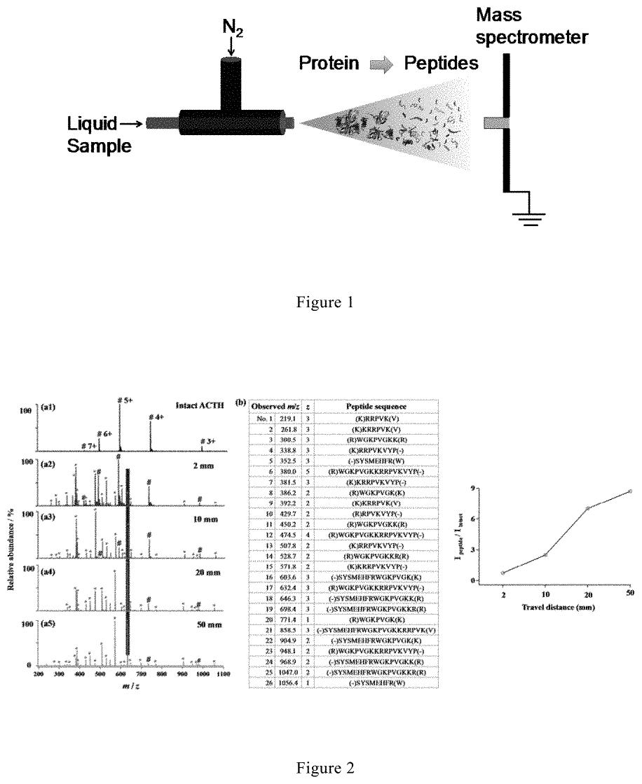

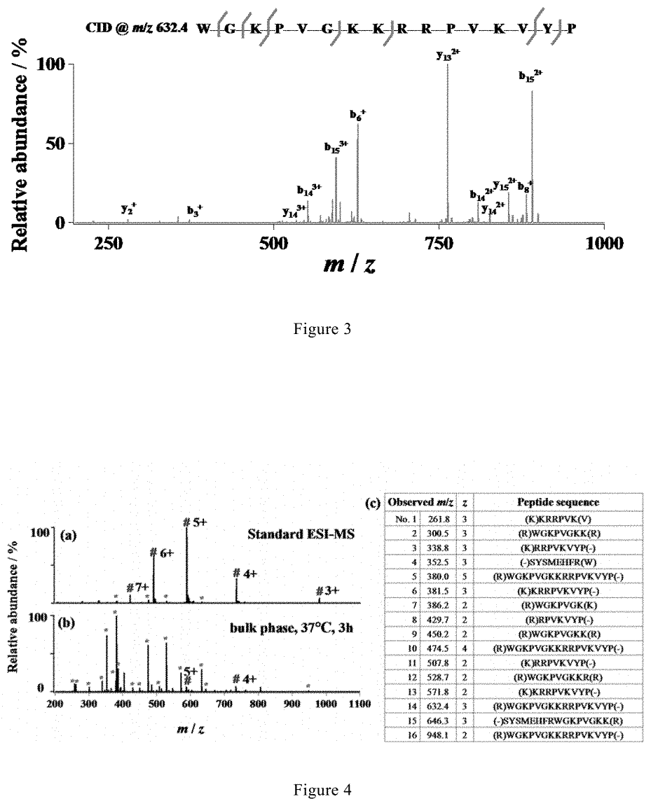

As shown in FIG. 2, digestion progressed as the travel time to the mass spectrometer inlet increased. The digestion efficiency was directly correlated with the reaction time in microdroplets, which can be seen by examining FIGS. 2(a2-a5), where the digestion yield of ACTH was sharply improved by increasing the travel distance from 2 mm to 20 mm. Twenty-six peptide fragment peaks were successfully identified, fully covering the whole sequence of ACTH, as listed in FIG. 2b. When the distance was increased to 50 mm, corresponding to a digestion time of 0.6 ms based on the previously reported microdroplet velocity of 80 m/s, digestion of ACTH was almost complete, with only tiny peaks from ACTH appearing in FIG. 2(a5). The dependence of digestion yield on travel distance of microdroplets was also clearly shown in FIG. 2c by comparing the peak intensity ratio of the most abundant peptide in FIG. 2(a2)-2(a5) at m/z 632.4 to the intensity sum of all the peaks from the intact multicharged ACTH, including (ACTH+3H)³⁺, (ACTH+4H)⁴⁺, (ACTH+5H)⁵⁺, (ACTH+6H)⁶⁺ and (ACTH+7H)⁷⁺. The amount of this chosen peptide increased with increasing travel distance. The sequence of this peptide was confirmed by tandem MS using collision induced dissociation (CID), as shown in FIG. 3.

### Comparative Example 1

ACTH Digestion with Standard ESI-MS.

ACTH was mixed with trypsin solution and infused into MS for digestion and detection with a standard ESI source. During this procedure, only very slight digestion of ACTH was observed.

As shown in FIG. 4a, owing to the bigger size of droplets generated by the commercial ESI source (which has a larger capillary inner diameter), only a slight amount of digestion was observed.

Because the standard ESI-MS causes negligible digestion acceleration (as shown in FIG. 4a, the same solution in bulk solution was first digested at 37° C. for 3 hours using a traditional procedure: 10 μM adrenocorticotropic hormone from human (ACTH, 1-24, Genscript, China) or 100 μg/mL proteins were denatured by heating at 95° C. for 5 min and then were incubated with 5 μg/mL of trypsin in a 5 mM NH₄HCO₃ buffer, pH 8, under 37° C. Aliquots of 100 μL were taken at different reaction time for freezing at −20° C. to stop the reaction. Then, the digestion products were recorded using standard ESI-MS.

For the analysis with MS, the samples were directly infused with a syringe at the flow rate of 10 μL/min and sprayed from commercial electrospray ionization (ESI) source with a needle of 500 μm in diameter and assisted with a sheath gas flow of 10 arbitrary units with a gas flow of 5 arbitrary units. The temperature of the MS inlet capillary was set at 275° C. and the ESI voltage was set as ±3 kV.

Compared with twenty-six peptide fragments fully covering the whole sequence of ACTH detected by microdroplet-MS in Example 1 (FIG. 2b), only 16 peptides were detected by using standard ESI-MS after 3 hours of bulk-phase digestion (FIGS. 4b and 4c).

### Example 2

Microdroplet-MS for Digestion and Analysis of Protein that Particularly Recalcitrant to Tryptic Digestion.

A stream of microdroplets was generated by infusing an aqueous sample solution containing myoglobin (10 μM) and trypsin (5 μg/mL) in 5 mM ammonia bicarbonate (NH₄HCO₃, pH 8) with a syringe at a flow rate of 10 μL/min into a homemade sprayer (with a capillary of 50 μm i.d and 148 μm o.d, as shown in FIG. 1).

The sample solution was sprayed from the tip of a fused silica capillary (148 μm o.d., 50 μm i.d., Polymicro Technologies, China) and assisted by a nebulizing gas of dry N₂ with a pressure of 120 psi. By placing the sprayer in front of a high-resolution mass spectrometer (LTQ Orbitrap Elite, Thermo Scientific, San Jose, Calif.) at a proper position, the microdroplets were directed into MS for real-time detection when applying a positive high voltage of +3 kV (BOHER H V, Genvolt, U.K.) to the sprayer. The MS inlet capillary was maintained at 275° C. and capillary voltage at 0 V. No other source gases were used when digestion was performed in microdroplets.

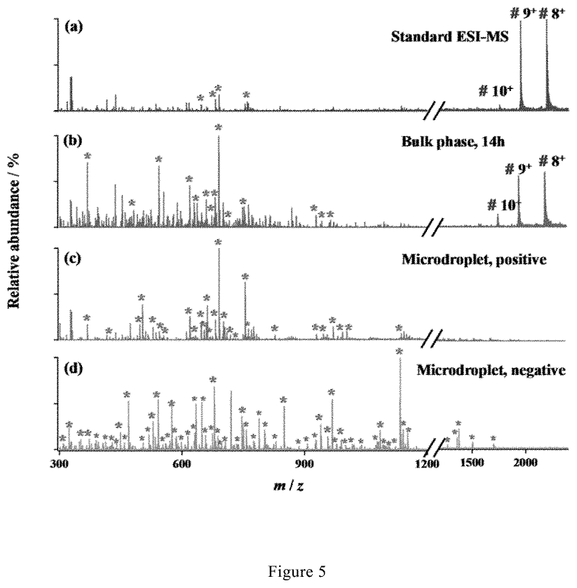

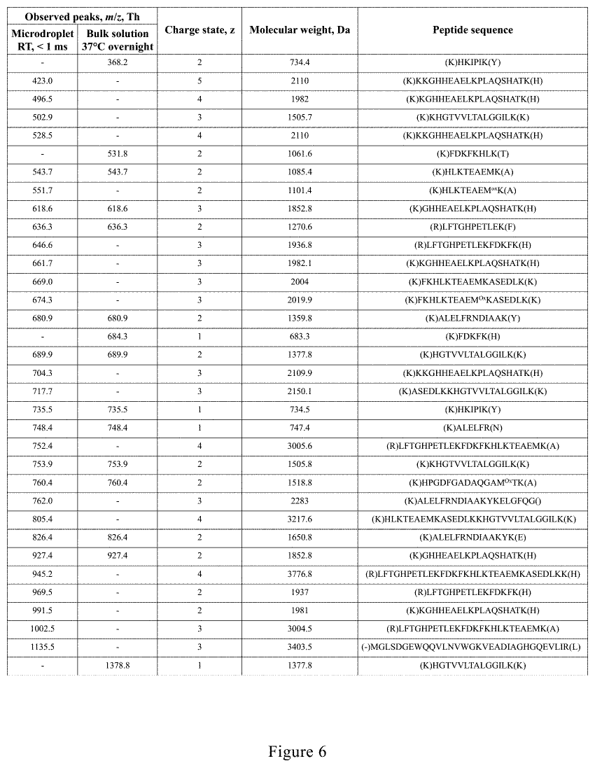

As shown in FIG. 5c and FIG. 6, 31 peaks corresponding to 19 peptides were identified, and high sequence coverage of 86% was obtained.

To find the lost sequence, a negative high voltage of −3 kV was applied instead of the positive high voltage owing to the more compatible pH value with the tryptic digestion.

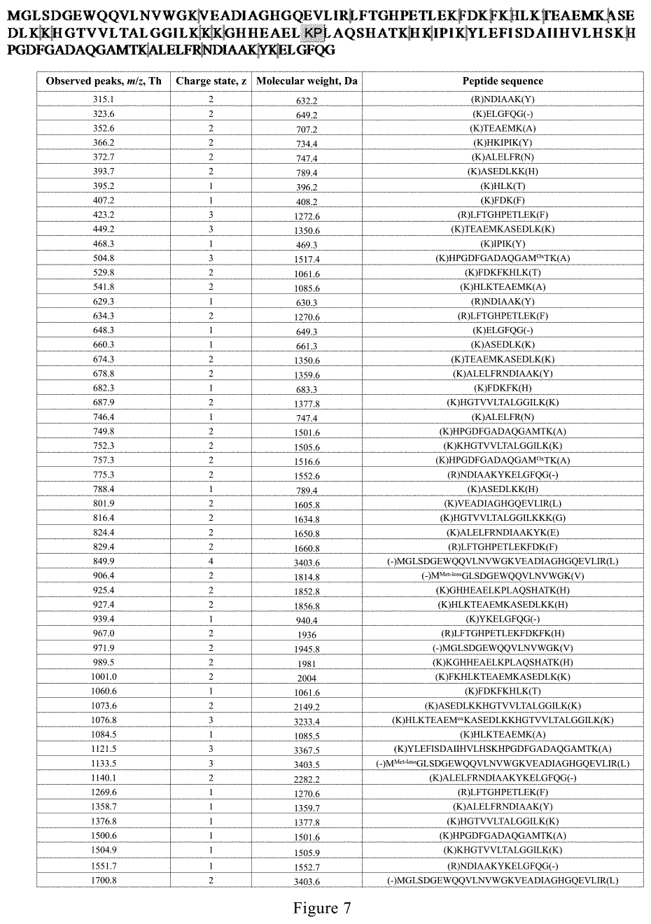

As shown in FIG. 5d and FIG. 7, surprisingly, 55 peaks corresponding to 38 peptides were identified. This corresponds to 100% sequence coverage. By matching the experimental results with the results of an in silico digest, all theoretical-cleavable peptide bonds after K and R except when following by proline due to the steric hindrance were found to be broken. Moreover, it is shown that trypsin digested nearly all proteins in the initial starting solution under microdroplet-MS.

### Comparative Example 2

Myoglobin Digestion with Standard ESI-MS.

Myoglobin was mixed with trypsin solution and infused into MS for digestion and detection with a standard ESI source. During this procedure, only very slight digestion of myoglobin was observed.

As shown in FIG. 5a, only a slight amount of digestion was observed.

Because the standard ESI-MS causes negligible digestion acceleration (as shown in FIG. 5a), the same solution in bulk solution was first digested at 37° C. for 14 hours using a traditional procedure: 10 μM adrenocorticotropic hormone from human (ACTH, 1-24, Genscript, China) or 100 μg/mL proteins were denatured by heating at 95° C. for 5 min and then were incubated with 5 μg/mL of trypsin in a 5 mM NH₄HCO₃ buffer, pH 8, under 37° C. Aliquots of 100 μL were taken at different reaction time for freezing at −20° C. to stop the reaction. Then, the digestion products were recorded using standard ESI-MS.

For the analysis with MS, the samples were directly infused with a syringe at the flow rate of 10 μL/min and sprayed from commercial electrospray ionization (ESI) source with a needle of 500 μm in diameter and assisted with a sheath gas flow of 10 arbitrary units with a gas flow of 5 arbitrary units. The temperature of the MS inlet capillary was set at 275° C. and the ESI voltage was set as ±3 kV.

Compared with 38 peptides corresponding to 100% sequence coverage of myoglobin detected by microdroplet-MS in Example 2 (FIG. 5d), only 13 peptides corresponding to 60% sequence coverage were detected by using standard ESI-MS after 14 hours of bulk-phase digestion (FIG. 5b), demonstrating that the time for myoglobin digestion are dramatically reduced by microdroplet-MS from overnight to less than 1 ms.

### Example 3

Microdroplet-MS for Digestion and Analysis of Cytochrome c.

According to the same method as described in Example 1 and Example 2, microdroplet-MS was further used for the digestion and analysis of cytochrome c under a positive voltage of +3 kV.

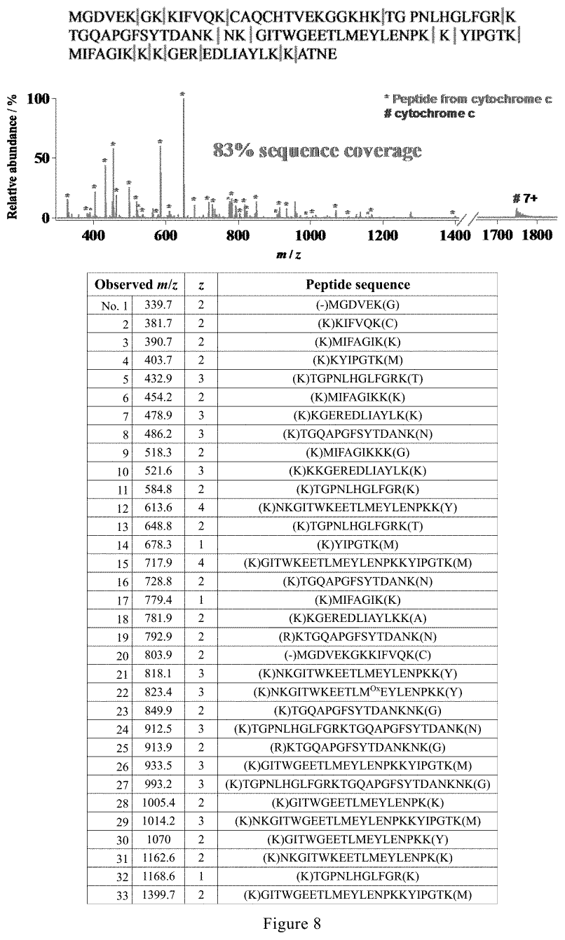

As shown in FIG. 8, 33 peptide fragments corresponding to 83% sequence coverage of cytochrome c was successfully identified. The results demonstrate that microdroplet-MS is a universal tool for protein digestion.

### Example 4

According to the same method as described in Example 1 and Example 2, synthetic peptide sample (LYAA-[DTR]-LYAVR, 10-μM in 5 mM NH₄HCO₃) with a very low kinetic constant (0.24×10⁻³s⁻¹) was subjected to enzymatic digestion by microdroplet-MS under a high voltage of +3 kV.

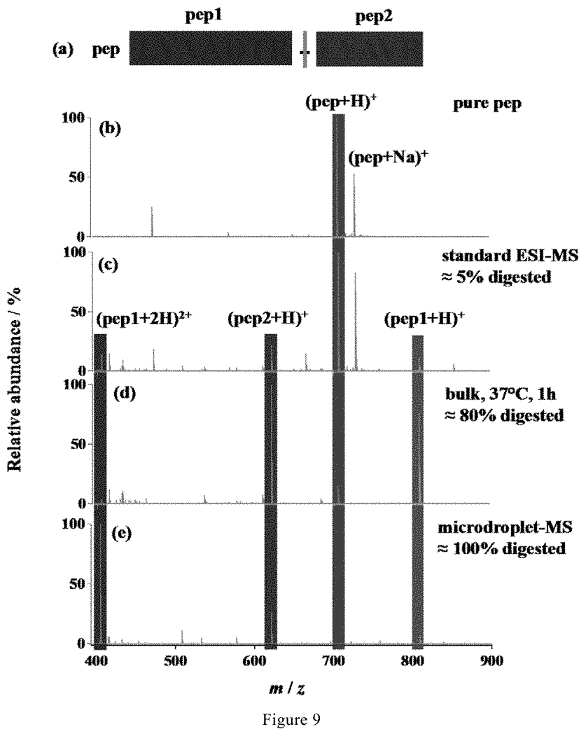

As shown in FIG. 9e, microdroplets could completely cleave the synthetic peptide sample (100% digestion), which is mainly contributed to the remarkable acceleration of proteolysis speed by microdroplets despite of the negative influence of acidic microdroplets on enzymatic digestion.

### Comparative Example 3

Analysis of a Synthetic Peptide Sample (LYRA-[DTR]YAVR) with Standard ESI-MS.

A synthetic peptide sample (LYAA-[DTR]-LYAVR) was mixed with trypsin solution and infused into MS for digestion and detection with a standard ESI source. During this procedure, only slight digestion of peptide was observed.

As shown in FIG. 9c, only about 5% digestion was observed by standard ESI-MS alone.

Accordingly, the same solution in bulk solution was digested at 37° C. for 1 hour using the traditional procedure as described in Comparative Examples 1 and 2. Then, the digestion products were recorded using standard ESI-MS.

As shown in FIG. 9d, after digestion at 37° C. for 1 hour, about 80% digestion was observed by standard ESI-MS, which is still much less than the 100% digestion obtained by microdroplet-MS in Example 4 (FIG. 9e).

### Example 5

pH Range that Allows Microdroplet-MS to Maintain Excellent Acceleration Effect for Proteolysis.

It is known that a higher H⁺ concentration could facilitate acid hydrolysis of proteins and a higher OH⁻ concentration would promote trypsin activity.

The inventors also examined the proper pH range that allows microdroplet-MS to maintain excellent acceleration effect for the proteolysis. The synthetic peptide (LYAA-[DTR]-LYAVR) in 5 mM NH₄HCO₃ with different pH values (i.e., pH 3˜4, 5, and 11) was subjected to microdroplet-MS analysis with a positive voltage.

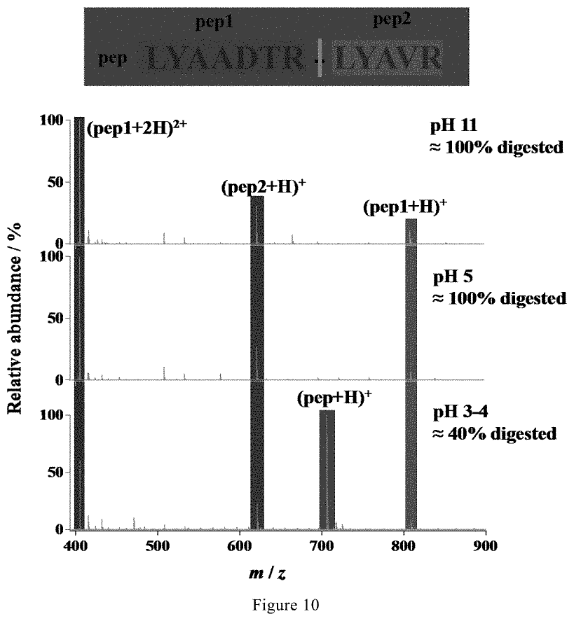

As shown in FIG. 10, it is found that when the pH is lowered than 4, the accelerated digestion by microdroplet-MS was inhibited, with only 40% of the synthetic peptide being digested. When the pH is up to 11, the microdroplet-MS could still digest the synthetic peptide completely. Therefore, the proper pH range can be determined as 4˜11.

### Example 6

Practicability in Proteomics

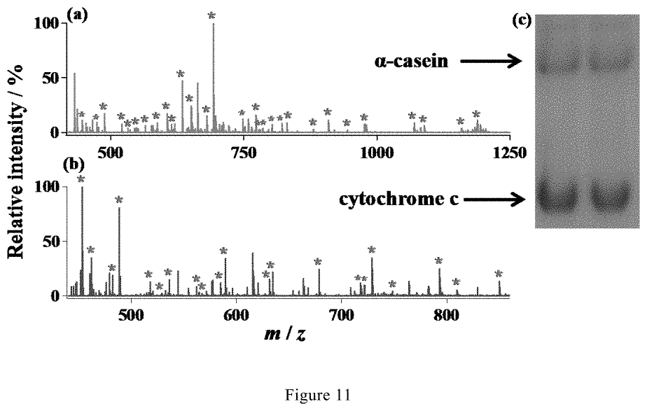

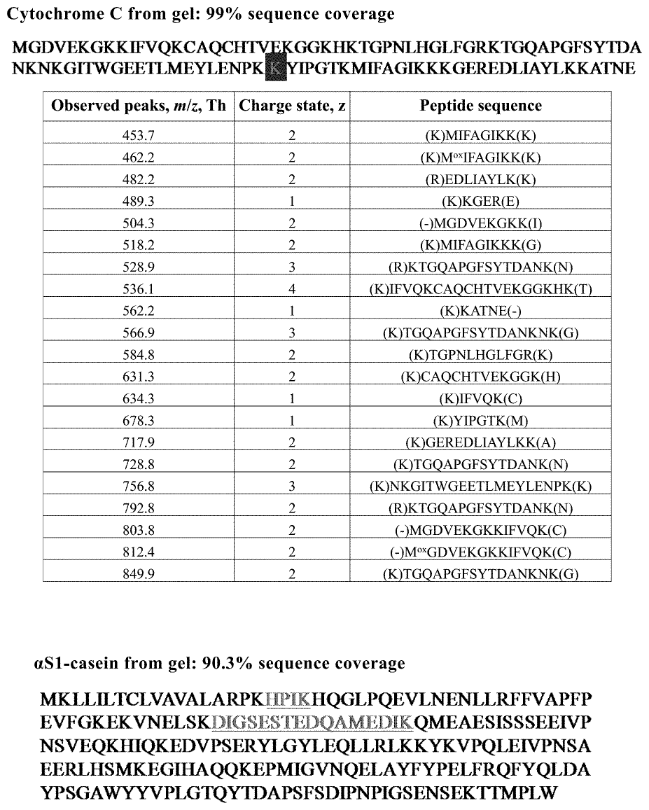

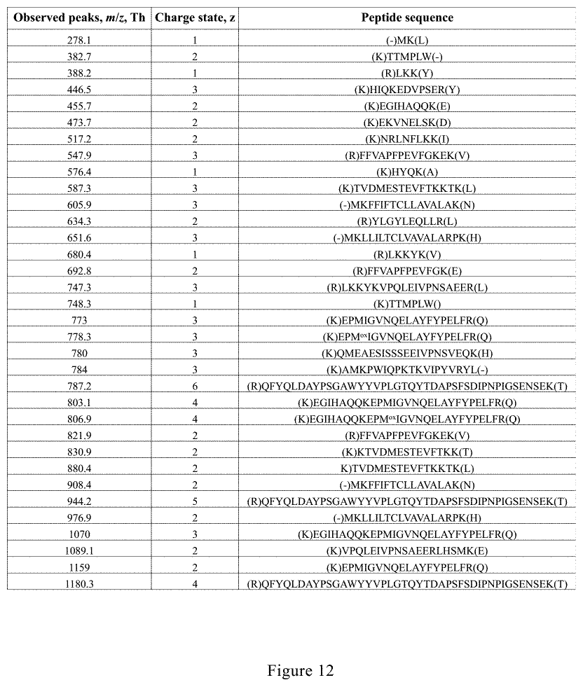

To further demonstrate the practicability in proteomics study, protein mixture containing cytochrome c and α-casein were separated first by 15.5% SDS polyacrylamide gel electrophoresis (PAGE), as shown in FIG. 11(c). Then, the Coomasie blue-stained protein bands were excised from the gel and subjected to digestion by microdroplet MS after a treatment procedure as described below.

For protein separation by gel electrophoresis, 10 μL of sample solution containing cytochrome c (1 mg/mL) and α-casein (1 mg/mL) were loaded onto 15.5% SDS-PAGE gels. All the setups and reagents for gel electrophoresis were purchased from Sangon (Shanghai, China). Electrophoresis was carried out at 200 V for 1 h at RT. Low range protein ladder was used as the size marker. The gel was stained by Coomasie blue and then destained by a solution containing 30% ethanol and 12.5% acetic acid in H₂O. The stained protein bands were excised from the gels and grund into tiny pieces for efficient protein extraction with a commercial kit (Sangon, Shanghai). The sample was sonicated in an ultrasonic water bath for 30 min until the gel pieces turned opaque. The extracted proteins were further purified by performing precipitation in pure acetone (99.9%, Adamas, China) at −20° C. for 3 times and then desalting by a centrifugal filter (Amicon Ultra-0.5, Millipore, USA) with a nominal molecular weight limit of 10 kDa. The purified protein samples were diluted in 5 mM NH₄HCO₃ buffer, pH 8 and further submitted to digestion by microdroplet MS.

As a result, the mass spectra gave high sequence coverage of 90.3% and 99% for αS1-casein and cytochrome c, respectively, in FIG. 11(a)-(b) and FIG. 12. The time for protein extraction from gel and off-gel digestion with microdroplet MS has been significantly reduced from overnight by in-gel digestion to be less than 1 h.

The above elucidate the great advance and potential significance of microdroplet-MS in proteomics, including the dramatic decrease in digestion time from overnight to less than a millisecond, complete cleavage of peptide bonds at the C-terminal side of lysine or arginine residues except when followed by proline, and the increase of sequence coverage from 60% to 100%.

### CITATION LIST

1. Shan, B., Yates, J. R., Baek, M. C., Zhang, Y. & Fonslow, B. R. Protein Analysis by Shotgun/Bottom-up Proteomics. Chem. Rev. 113, 2343-2394 (2013).
2. Basile, F. & Hauser, N. Rapid online nonenzymatic protein digestion combining microwave heating acid hydrolysis and electrochemical oxidation. Anal. Chem. 83, 359-367 (2011).
3. Yan, X., Bain, R. M. & Cooks, R. G. Organic Reactions in Microdroplets: Reaction Acceleration Revealed by Mass Spectrometry. Angew. Chemie—Int. Ed. 55, 12960-12972 (2016).

The invention has been described in terms of particular embodiments found or proposed to comprise specific modes for the practice of the invention. Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A method of protein sequence analysis, comprising the following steps:
a) forming a solution containing protein into microdroplets having a size small enough to result in acceleration of protein digestion;
b) introducing the microdroplets into a mass spectrometer MS for real-time detection;
c) obtaining analysis result of the protein from the mass spectrometer MS;
wherein the protein is fully digested in the microdroplets before entering the mass spectrometer MS.

2. The method according to claim 1, wherein the method does not require any pre-treatment step for enzymatic digestion.

3. The method according to claim 1, wherein the protein is recalcitrant to tryptic digestion.

4. The method according to claim 1, wherein the pH value of the solution containing protein is in the range of 4˜11.

5. The method according to claim 4, wherein the pH value of the solution containing protein is about 8.

6. The method according to claim 1, wherein in step a), the microdroplets are formed by a spray-based ionization method.

7. The method according to claim 1, wherein in step a), the microdroplets are formed by means of capillary of a sprayer.

8. The method according to claim 7, wherein the sprayer is applied with pressurized nebulizing N₂ gas at a pressure of 120 psi or more.

9. The method according to claim 7, wherein the sprayer is applied with a positive or negative voltage of 3 kV or above.

10. The method according to claim 1, wherein the microdroplets travel a distance of 10 mm to 10 cm before entering the mass spectrometer.

11. The method according to claim 10, wherein the microdroplets travel a distance of 50 mm before entering the mass spectrometer.

12. The method according to claim 10, wherein the microdroplets travel a distance of 2 cm before entering the mass spectrometer.

13. A microdroplet-MS device for protein sequence analysis, comprising:
a microdroplet-producing unit, and
a mass spectrometer MS unit that is directly coupled with the microdroplet-producing unit,
wherein the microdroplet-producing unit forms a protein-containing solution into microdroplets having a size small enough to result in acceleration of protein digestion.

14. The microdroplet-MS device according to claim 13, wherein the microdroplet-producing unit includes a sprayer.

15. The microdroplet-MS device according to claim 14, wherein the sprayer is provided with a capillary having an inner diameter of 50˜100 μm.

16. The microdroplet-MS device according to claim 14, wherein the sprayer is applied with a positive or negative voltage of 3 kV or above.

17. The microdroplet-MS device according to claim 14, wherein the travel distance of microdroplets from the sprayer to the mass spectrometer is in the range of 10 mm to 10 cm.

18. The microdroplet-MS device according to claim 14, wherein the travel distance of microdroplets from the sprayer to the mass spectrometer is 50 mm.

19. The microdroplet-MS device according to claim 14, wherein the travel distance of microdroplets from the sprayer to the mass spectrometer is 2 cm.

20. Use of microdroplets for improving protein sequencing by accelerating enzymatic digestion, wherein the microdroplets are formed from protein-containing solution and have a size small enough to result in acceleration of protein digestion.
